SEQUENCE LISTING (34 sequences)

SEQ ID NO: 1 - DNA, 21 bp, synthetic construct
  misc_feature 1..21: note = primer
  source 1..21: mol_type = other DNA
tcggcggttatttgtatttgc

SEQ ID NO: 2 - DNA, 20 bp, synthetic construct
  misc_feature 1..20: note = primer
  source 1..20: mol_type = other DNA
cgtcgaaaacaaccgaaaca

SEQ ID NO: 3 - DNA, 16 bp, synthetic construct
  misc_feature 1..16: note = primer
  source 1..16: mol_type = other DNA
gttcggggttacgcgc

SEQ ID NO: 4 - DNA, 18 bp, synthetic construct
  misc_feature 1..18: note = primer
  source 1..18: mol_type = other DNA
ttcgcctcactctccgct

SEQ ID NO: 5 - DNA, 20 bp, synthetic construct
  misc_feature 1..20: note = primer
  source 1..20: mol_type = other DNA
ttcgggagttagttcgcgtt

SEQ ID NO: 6 - DNA, 19 bp, synthetic construct
  misc_feature 1..19: note = primer
  source 1..19: mol_type = other DNA
accgccgaacatcctacga

SEQ ID NO: 7 - DNA, 18 bp, synthetic construct
  misc_feature 1..18: note = primer
  source 1..18: mol_type = other DNA
gcgttgaagtcggggttc

SEQ ID NO: 8 - DNA, 24 bp, synthetic construct
  misc_feature 1..24: note = primer
  source 1..24: mol_type = other DNA
cccgtacttcgctaactttaaacg

SEQ ID NO: 9 - DNA, 18 bp, synthetic construct
  misc_feature 1..18: note = primer
  source 1..18: mol_type = other DNA
gcggtcgttcggcgtgtc

SEQ ID NO: 10 - DNA, 21 bp, synthetic construct
  misc_feature 1..21: note = primer
  source 1..21: mol_type = other DNA
aaacgaaccgaaccgaaaacg

SEQ ID NO: 11 - DNA, 19 bp, synthetic construct
  misc_feature 1..19: note = primer
  source 1..19: mol_type = other DNA
gaaccaaaacgctccccat

SEQ ID NO: 12 - DNA, 27 bp, synthetic construct
  misc_feature 1..27: note = primer
  source 1..27: mol_type = other DNA
ttatatgtcggttacgtgcgtttatat

SEQ ID NO: 13 - DNA, 21 bp, synthetic construct
  misc_feature 1..21: note = primer
  source 1..21: mol_type = other DNA
tcggcgtacgtaagagtttgg

SEQ ID NO: 14 - DNA, 24 bp, synthetic construct
  misc_feature 1..24: note = primer
  source 1..24: mol_type = other DNA
atctcccgatcctaaacgataaaa

SEQ ID NO: 15 - DNA, 17 bp, synthetic construct
  misc_feature 1..17: note = primer
  source 1..17: mol_type = other DNA
agtcgggcggttcggtt

SEQ ID NO: 16 - DNA, 17 bp, synthetic construct
  misc_feature 1..17: note = primer
  source 1..17: mol_type = other DNA
caaaacgacgcgcaact

SEQ ID NO: 17 - DNA, 21 bp, synthetic construct
  misc_feature 1..21: note = primer
  source 1..21: mol_type = other DNA
ggttattttgcgagcggtttc

SEQ ID NO: 18 - DNA, 24 bp, synthetic construct
  misc_feature 1..24: note = primer
  source 1..24: mol_type = other DNA
cgaatactctaattccacgcgact

SEQ ID NO: 19 - DNA, 22 bp, synthetic construct
  misc_feature 1..22: note = primer
  source 1..22: mol_type = other DNA
ttacgtgttattgtcggggatt

SEQ ID NO: 20 - DNA, 29 bp, synthetic construct
  misc_feature 1..29: note = primer
  source 1..29: mol_type = other DNA
atctattcctaatacctaaaaacgaaacg

SEQ ID NO: 21 - DNA, 34 bp, synthetic construct
  misc_feature 1..34: note = primer
  source 1..34: mol_type = other DNA
cgtatgttattagtattttattagttgttcgttc

SEQ ID NO: 22 - DNA, 21 bp, synthetic construct
  misc_feature 1..21: note = primer
  source 1..21: mol_type = other DNA
cgctcgacaacactacactcg

SEQ ID NO: 23 - DNA, 17 bp, synthetic construct
  misc_feature 1..17: note = primer
  source 1..17: mol_type = other DNA
ggttattgggtttcgcg

SEQ ID NO: 24 - DNA, 24 bp, synthetic construct
  misc_feature 1..24: note = primer
  source 1..24: mol_type = other DNA
gtactaacgtcgaccttaaaacga

SEQ ID NO: 25 - DNA, 28 bp, synthetic construct
  misc_feature 1..28: note = primer
  source 1..28: mol_type = other DNA
ggtaattaatttggatttacgtcgttat

SEQ ID NO: 26 - DNA, 32 bp, synthetic construct
  misc_feature 1..32: note = primer
  source 1..32: mol_type = other DNA
cgtaaaaatataaacgatacgtatacctacgt

SEQ ID NO: 27 - DNA, 25 bp, synthetic construct
  misc_feature 1..25: note = primer
  source 1..25: mol_type = other DNA
tggtgatggaggaggtttagtaagt

SEQ ID NO: 28 - DNA, 27 bp, synthetic construct
  misc_feature 1..27: note = primer
  source 1..27: mol_type = other DNA
aaccaataaaacctactcctcccttaa

SEQ ID NO: 29 - DNA, 23 bp, synthetic construct
  misc_feature 1..23: note = primer
  source 1..23: mol_type = other DNA
cgaggtcgatcgttagttcgttc

SEQ ID NO: 30 - DNA, 23 bp, synthetic construct
  misc_feature 1..23: note = primer
  source 1..23: mol_type = other DNA
ccaacgactaactcgaaaacgcg

SEQ ID NO: 31 - DNA, 25 bp, synthetic construct
  misc_feature 1..25: note = primer
  source 1..25: mol_type = other DNA
cgttttagtcgtcggttgttagttt

SEQ ID NO: 32 - DNA, 18 bp, synthetic construct
  misc_feature 1..18: note = primer
  source 1..18: mol_type = other DNA
tcccgaacccgctctctt

SEQ ID NO: 33 - DNA, 21 bp, synthetic construct
  misc_feature 1..21: note = primer
  source 1..21: mol_type = other DNA
gatttttattaagcgggcgtc

SEQ ID NO: 34 - DNA, 25 bp, synthetic construct
  misc_feature 1..25: note = primer
  source 1..25: mol_type = other DNA
cttttcctatcacaaaaataatccg

(19) United States
(12) Patent Application Publication
(10) Pub. No.: US 2024/0229153 A9
(43) Pub. Date: Jul. 11, 2024

(54) Detection of Cancer in Urine

(71) Applicant: Nanomed Diagnostics B.V., Enschede (NL)

(72) Inventors: Willem Frederik Rurup, Best (NL); Loes Irene Segerink, Enschede (NL); Roderik Adriaan Kraaijenhagen, Amstelveen (NL); Herbert Michael Pinedo, Amsterdam (NL); Albert Van Den Berg, Nijverdal (NL); Renske Daniela Maria Steenbergen, Enschede (NL); Jacobus Adrianus Nieuwenhuijzen, Amsterdam (NL); Idris Bahce, Enschede (NL); Geert Kazemier, Enschede (NL)

(21) Appl. No.: 18/497,332

(22) Filed: Oct. 30, 2023

Related U.S. Application Data
(63) Continuation of application No. 16/089,222, filed on Sep. 27, 2018, now Pat. No. 11,821,040.

(30) Foreign Application Priority Data
Apr. 1, 2016 (EP) 16163585.9

(51) Int. Cl. C12Q 1/6886; C12Q 1/6806; C12Q 1/686

(52) U.S. Cl. CPC C12Q 1/6886; C12Q 1/6806; C12Q 1/686; C12Q 2600/112; C12Q 2600/118; C12Q 2600/154; C12Q 2600/156; C12Q 2600/16

(57) ABSTRACT

The invention provides in vitro methods of determining whether an individual has a pre-cancer or cancer comprising determining the presence or absence of one or more methylation markers of a methylation marker set in a urine sample of said individual; and determining whether the individual has pre-cancer or cancer based on the detection of the presence or absence of said one or more methylation markers in the urine sample, wherein the presence of said one or more methylation markers indicates that the individual has pre-cancer or cancer. The invention further provides methods for typing pre-cancer or cancer based on the presence or absence of one or more methylation markers of a methylation marker set in a urine sample.

### CROSS-REFERENCE TO RELATED APPLICATIONS

This application is a continuation of U.S. patent application Ser. No. 16/089,222, filed Sep. 27, 2018, pending, which is a national phase entry under 35 U.S.C. § 371 of International Patent Application PCT/NL2017/050199, filed Mar. 31, 2017, designating the United States of America and published in English as International Patent Publication WO WO2017/171548 on Oct. 5, 2017, which claims the benefit under Article 8 of the Patent Cooperation Treaty to European Patent Application Serial No. 16163585.9, filed Apr. 1, 2016, the entireties of which are hereby incorporated by reference.

### INCORPORATION BY REFERENCE

The ST.26 XML Sequence listing named “10273 US 2023-10-30-ST26 Sequence listing”, created on Oct. 30, 2023, and having a size of 43,589 bytes, is hereby incorporated herein by this reference in its entirety.

The invention relates to the field of analyzing urine samples. In particular the invention relates to the identification of cancerous nucleic acid in the urine of individuals, in particular methylated nucleic acid.

Cancer is nearly always diagnosed by an expert who has looked at cell or tissue samples under a microscope. In some cases, tests done on the cells' proteins, DNA, and RNA can help tell doctors if there is cancer. These test results are important when choosing the best treatment options.

Such tests are typically performed when an individual registers itself with complaints. In such cases, if it is cancer, chances are that the cancer has already progressed to such an extent that simple surgery is not curative or the chance of metastasis is significant resulting in a need for invasive treatments with chemotherapy and/or radiation and a poor general prognosis.

On the other hand, when the complaints are not caused by cancer the patient is left in uncertainty for a considerable amount of time waiting for the results of the biopsy.

There is thus a need for a simple, quick and non-invasive method to determine whether the individual has cancer and, if it is cancer, what type of cancer it is.

The present invention provides means and methods for the detection and the typing of cancer and pre-cancer in urine samples. The means and methods detects nucleic acid, and in particular DNA in the urine and detects and types cancer or pre-cancer by analyzing the methylation state of the detected nucleic acid.

It is known in the art that certain types of cancer and pre-cancer are associated with the methylation of certain genes. Methylation of DNA is a natural process and typically occurs when cells differentiate to specialized cells in the body. DNA methylation is a process by which methyl groups are added to DNA. Methylation has been shown to alter the behavior and the function of certain DNAs. When located in a gene promoter, DNA methylation typically acts to repress gene transcription. DNA methylation is essential for normal development and is associated with a number of key processes including genomic imprinting, X-chromosome inactivation, repression of repetitive elements and ageing. In disease it has been associated with cancer.

In eukaryotes, methylation is restricted to one of the DNA's four nucleotides. The incidence of cytosine DNA methylation differs between species. DNA methylation typically occurs in a CpG dinucleotide context. Other forms of methylation have been detected, mostly in embryonic stem cells or neural development.

Between 60% and 90% of all CpGs are methylated in mammals. Methylated C residues spontaneously deaminate to form T residues over time, which is thought to be the reason for the relative under-representation of CpG dinucleotides in the human genome.

Unmethylated CpGs are often grouped in clusters called CpG islands, which are present in the 5′ regulatory regions of many genes. In cancer and pre-cancer, gene promoter CpG islands acquire abnormal hypermethylation, which results in transcriptional silencing that can be inherited by daughter cells following cell division. Alterations of DNA methylation have been recognized as an important component of cancer development. Hypomethylation, in general, arises earlier and is linked to chromosomal instability and loss of imprinting, whereas hypermethylation is associated with promoters.

The glomerular filtration barrier causes substances of low molecular weight (<5500 Da) and small effective molecular radius (e.g., water, urea and glucose) to appear in the filtrate in the same concentration as in plasma. Larger and larger macromolecules are increasingly restricted from passage so that only traces of plasma albumin (69 kDa) are normally present in the glomerular filtrate. Other factors such as the radius and electrical charge also influence the appearance of substances in the filtrate. Measurements suggest that the glomerular filtration restricts the passage of anions but enhances the passage of cations. This conclusion was based in part on the observation that longer and/or more negatively charged sugar-chains (dextrans) appear to pass with lower efficiency.

The function of renal tubules is to recover most of the fluid and solutes filtered at the glomerulus. If the fluid was not recovered, the kidney would excrete the volume of the entire blood plasma in less than half an hour. In the tubules NaCl, NaHCO₃, filtered nutrients (e.g., glucose and amino acids), divalent ions (e.g., Ca²⁺, HPO₄²⁻, and SO₄²⁻), and water are reabsorbed. Finally, the proximal tubule secretes NH⁴⁺ and a variety of endogenous and exogenous solutes into the lumen making a concentrate with waste products, to compose the final urine (Boulpaep, et al, Medical Physiology, 2012, Philadelphia: Saunders).

Among the different components of urine it was found that some DNA is also present. The majority of the DNA in urine is found in cells. According to a manufacturer of DNA isolation kits specific for urine (Zymoresearch), the total amount of recoverable DNA is approximately 5-25 μg/L urine.

### SUMMARY OF THE INVENTION

The invention provides an in vitro method determining whether an individual detecting has a pre-cancer or cancer comprising
determining the presence or absence of one or more methylation markers of a methylation marker set in a urine sample of said individual; and
determining whether the individual has pre-cancer or cancer based on the detection of the presence of said one or more methylation markers in the urine sample, wherein the presence or absence of said one or more methylation markers indicates that the individual has pre-cancer or cancer and the absence of the methylation markers indicates that the individual does not have pre-cancer or cancer.

The invention also provides an in vitro method of determining a type of pre-cancer or cancer in an individual that has cancer comprising
determining the presence or absence of one or more methylation markers of a methylation marker set in a urine sample of said individual; and
determining pre-cancer or cancer type(s) based on the detection of the presence or absence of said one or more methylation markers in the urine sample, wherein the presence or absence of said one or more methylation markers is indicative for one or more types of pre-cancer or cancer of said individual.

The invention further provides a kit comprising means for the detection of one or more methylation markers in a methylation marker set wherein the set comprises at least one methylation marker selected from the group consisting of the methylation markers identified in Table 9 and wherein the set further comprises an nucleic acid extraction buffer for extraction of nucleic acid from urine.

Also provided is a nucleic acid amplification device comprising at least two nucleic acid amplification primers for amplifying nucleic acid of one or more methylation markers from a methylation marker set.

The invention further provides the use of kit according to the invention or a device according to the invention, for determining the type of pre-cancer or cancer of an individual with cancer from a urine sample of the individual.

The invention further provides the use of kit according to the invention or a device according to the invention, for detecting a pre-cancer or cancer in an individual from a urine sample of the individual.

### DETAILED DESCRIPTION OF THE INVENTION

The invention provides in vitro methods of determining whether an individual has a pre-cancer or cancer in an individual. The present invention further provides in vitro methods of determining a type of a pre-cancer or cancer in an individual that has pre-cancer or cancer. The methods comprise determining the presence or absence of one or more methylation markers of a methylation marker set in a urine sample of said individual. Pre-cancer and cancer types can be determined from the detection of the presence or absence of one or more particular methylation markers in the urine of the individual. The presence and/or the absence of certain methylation marker(s) is indicative for a type of pre-cancer or cancer the individual is likely to have. The presence or absence of one or more methylation markers indicates that the individual has pre-cancer or cancer and the absence of the methylation markers indicates that the individual does not have pre-cancer or cancer.

Various pre-cancers and cancers can be detected with the invention. Whereas some methylation markers are always detected, irrespective of the presence or absence of pre-cancer or cancer, some methylation markers are only detected in the urine when the individual has pre-cancer or cancer. Some cancer methylation markers are detected with various types of pre-cancer or cancer and are thus suited for determining whether the individual has pre-cancer or cancer as such, and (when combined with other methylation markers) also for typing cancer.

A precancerous condition or premalignant condition, sometimes called a potentially precancerous condition or potentially premalignant condition, is a state of disordered morphology of cells that is associated with an increased risk of cancer. If left untreated, these conditions may lead to cancer. Such conditions are usually dysplasia or benign neoplasia. Sometimes the term “pre-cancer” is used to describe carcinoma in situ, which is a noninvasive cancer that has not progressed to an aggressive, invasive stage. Not all carcinoma in situ will progress to invasive disease.

Pre-cancer (also referred to as premalignant) lesions are morphologically atypical tissue which appears abnormal under microscopic examination, and in which cancer is more likely to occur than in its apparently normal counterpart. Examples of pre-cancers are actinic keratosis; Barrett's esophagus; atrophic gastritis; dyskeratosis congenital; sideropenic dysphagia; lichen planus; oral submucous fibrosis; solar elastosis; cervical dysplasia; leukoplakia; polyposis coli and erythroplakia. In a preferred embodiment the pre-cancer is polyposis coli. Cancers that can be detected and/or types comprise and preferably consist of lung cancer, preferably non-small cell lung cancer, cervical cancer, prostate cancer, ovarian carcinoma, breast cancer, including ductal carcinoma in situ, head and neck cancer, Urothelial cell carcinoma such as bladder cancer, Hematological malignancies such as but not limited to myeloma, leukemia, lymphoma and myelodysplastic syndrome, a cancer of the gastrointestinal tract (GI tract) and accessory organs of digestion, including but not limited to the esophagus, stomach, biliary system, pancreas, small intestine, large intestine, and rectum. In a preferred embodiment the cancer that is to be detected comprises lung cancer, preferably non-small cell lung cancer, breast cancer, head and neck cancer, Hematological malignancies such as but not limited to myeloma, leukemia, lymphoma and myelodysplastic syndrome, a cancer of GI tract or accessory organs of digestion, including but not limited to the esophagus, stomach, biliary system, pancreas, small intestine, large intestine, rectum, gall bladder, liver and anus. Cancer in the present invention indicates primary cancer and metastasized cancer.

It is preferred that the pre-cancer or cancer is a pre-cancer or cancer of which the nucleic acid has had to pass the kidney to arrive in the urine. Even in a carefully collected urine sample, cancers such as Urothelial cell carcinoma such as bladder cancer or the pre-cancerous precursor stages thereof can shed nucleic acid directly into the urine without having to pass through the kidney. Pre-cancer lesions or cancer can be detected by detecting at least some of the methylation markers associated with urine of individuals in which the pre-cancerous state has progressed to cancer. In a preferred embodiment the cancer is not Urothelial cell carcinoma, cervical cancer or prostate cancer, or a pre-cancer stage thereof. In a preferred embodiment the cancer is not bladder cancer or a pre-cancer stage thereof.

Typing of a pre-cancer or cancer typically involves determining the tissue of origination of the cancer. For instance, bladder cancer that has metastasized to the lung is typically typed as bladder cancer. Typing can include determining whether a cancer is likely to have metastasized, this is optional. The typing can also result in the determination that the individual has one or more of several types of pre-cancer or cancer.

The individual is preferably a mammal, preferably a human.

Urine is a solution of which the concentration of components can vary in response to diet and the amount of liquid consumed. Among the various well known components of urine, nucleic acid is also present. Most of the DNA in urine is found in associated with cells. Commercial sources mention that the amount of DNA that can be recovered from urine is one average about 5-100 μg/L. In the present invention it is preferred that sample is a cell free urine sample. DNA can cross the kidney barrier in a polymer state, with fragments large enough (on average 150 bp) to be amplified with PCR. Cell free DNA in urine is on average about 2-100 μg/L. Not all urine samples contain sufficient DNA for analysis. Such samples are typically identified by performing a control. A suitable control is a PCR to determine the presence of a standard DNA such as actin. In cases of insufficient DNA it is often sufficient to collect a further sample of the individual. Alternatively the DNA can be concentrated from larger volumes of urine. It is also possible to concentrate urine by for example freeze drying.

Male gene fragments were shown in female urine after blood transfusion or in woman pregnant with males. In rodents injected human DNA sequences could also be amplified in the urine. Another study was able to capture 47-65 pg/μL (cell free) DNA per 1 mL sample (equaling 47-65 μg/L). Furthermore, colorectal tumor related mutations have been shown in free DNA derived from urine (Su et al 2008; Ann N Y Acad Sci. 2008 August; 1137: 197-206). Long-term storage of urine can affect the quality of DNA. Thus typically fresh urine samples are used in the present invention, typically samples are used within one day of sample, however, longer waiting times are possible upon appropriate processing of the urine sample (see for instance Hilhorst et al. 2013; BMC Nephrol, 2013. 14: p. 238). Other DNA preservations means and methods are also possible such as, for instance, commercially offered by Zymo research (urine conditioning buffer) which preserves DNA at room temperature for a number of days.

Aberrantly methylated cytosine at CpG dinucleotides is a widespread phenomenon in cancer. (Jones, P A and Laird, P W, “Cancer epigenetics comes of age,” Nat. Genet. 21: 163-167 (1999)). As a result of CpG island hypermethylation, chromatin structure in the promoter can be altered, preventing normal interaction with the transcriptional machinery. (Baylin, S B, et al. “Alterations in DNA methylation: A fundamental aspect of neoplasia,” Advances in cancer research (eds. G. F. Nande Woude and G. Klein), vol. 72: 141-196 (1998), Academic Press, San Diego, CA). When this occurs in genes that are relevant to growth inhibition, the resulting silencing of transcription could promote tumor progression. In addition, promoter CpG island hypermethylation has been shown to be a common mechanism for transcriptional inactivation of classic tumor suppressor genes and genes important for cell cycle regulation, and DNA mismatch repair. Methylation of cytosine, therefore, plays a significant role in control of gene expression, and a change in the methylation pattern or status is likely to cause disease. Differences in methylation at CpG dinucleotides between a normal state, a pre-cancer state and the cancer state of a cell are herein referred to as methylation markers. The detection of such a methylation marker in urine can be an indication that the individual has pre-cancer or cancer. Chromosomal DNA that has an increased methylation of CpG dinucleotides is also referred to as hypermethylated DNA. Hypermethylation in general is associated with the cancerous or pre-cancerous state of a cell. Methylation markers can be detected in various ways. Detection can be performed by using methylation assays capable of determining differential methylation levels within CpG sites between diseased cells or tissues and normal cells or tissues. Methylation-specific assays useful for this purpose include, for example, methylation-specific PCR, bisulfite genomic sequencing methods, methylation-specific primer extension methods, and other methods known in the art, and with high throughput or microarrays. Suitable methods for the detection of methylation markers are described in Shanmuganathan et al (2013) Journal of Molecular Diagnostics Vol 15: pp 17-26. This reference and the papers referred to therein in as far as they delineate methods for detection of methylation markers are incorporated by reference herein. A suitable method for next-generation bisulphite amplicon sequencing is described in Margolin et al (2016) Vol 18: pp 283-298, which is also incorporated by reference herein. A suitable device is disclosed in WO2009104967 which is also incorporated by reference herein.

Methylation markers of the present invention are typically not detected in the urine of individuals that do not have pre-cancer or cancer. Methylation markers of the present invention are only detected when the individual has pre-cancer or cancer. Different pre-cancers or cancers may be associated with different methylation markers in the urine. Such differences are exploited in the present invention for the typing of a pre-cancer or cancer. Various cancer types or pre-cancer types may be associated with the presence of the same methylation marker(s) in the urine of an individual presenting with the pre-cancer or cancer. Detection of such markers is indicative for the presence in the individual of one or more of the pre-cancers or cancers that the detected marker(s) are associated with. Such methylation markers are sometimes also referred to as common markers. Common markers are particularly suited to detect whether an individual has cancer or pre-cancer per se.

Detection of one methylation marker in the urine of an individual is indicative for the presence of a cancer or pre-cancer in the individual. Testing for more than one methylation marker in the urine typically allows for the more accurate determination of whether the individual has pre-cancer or cancer. Testing for more than one methylation marker in the urine typically allows for the more accurate typing of the pre-cancer or the cancer that the individual has. Markers that are common to many different types of cancer or pre-cancer are typically used in methods of the invention for determining whether an individual has cancer. Markers that are not shared by many pre-cancers or cancers, and preferably markers that are tumor-specific are typically used to type the pre-cancer or the cancer that the individual.

A method of the invention may simultaneously detect that an individual from which the urine was tested has cancer or pre-cancer and type the cancer or pre-cancer.

As used herein, the term “a polynucleotide primer/probe” refers to a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.) or sugar moiety, in addition, the bases in a primer/probe may be joined by a linkage other than a phosphodiester bond, so long as it does not interfere with hybridization. Thus, for example, primer/probes may be peptide nucleic acids in which the constituent bases are joined by peptide bonds rather than phosphodiester linkages. It will be understood by one of skill in the art that probes may bind target sequences lacking complete complementarity with the primer/probe sequence depending upon the stringency of the hybridization conditions. The primers/probes are preferably directly labeled as with isotopes, chromophores, lumiphores, chromogens, or indirectly labeled such as with biotin to which a streptavidin complex may later bind. By assaying for the presence or absence of the primer/probe, one can detect the presence or absence of the select sequence or subsequence.

As used herein, the term “methylation” refers to the covalent attachment of a methyl group at the C5-position of the nucleotide base cytosine within the CpG dinucleotides of gene regulatory region. The term “hypermethylation” refers to the methylation state corresponding to an increased presence of 5-methyl-cytosine (“5-mCyt”) at one or a plurality of CpG dinucleotides within a DNA sequence of a test sample, relative to the amount of 5-mCyt found at corresponding CpG dinucleotides within the DNA sequence of a sample of a normal individual or cell of the same type as the test sample. The term “methylation state” or “methylation status” or “methylation level” or “the degree of methylation” refers to the presence or absence of 5-mCyt at one or a plurality of CpG dinucleotides within a DNA sequence. As used herein, the terms “methylation status” or “methylation state” or “methylation level” or “degree of methylation” are used interchangeably.

As used here, the term “CpG islands” are short DNA sequences rich in the CpG dinucleotide and defined as sequences greater than 200 bp in length, with a GC content greater than 0.5 and an observed to expected ratio based on GC content greater than 0.6. See Gardiner-Garden and Frommer, “CpG islands in vertebrate genomes,” J Mol Biol 196(2): 261-282 (1987). CpG islands were associated with the 5′ ends of all housekeeping genes and many tissue-specific genes, and with the 3′ ends of some tissue-specific genes. A few genes contain both the 5′ and the 3′ CpG islands, separated by several thousand base pairs of CpG-depleted DNA. The 5′ CpG islands extended through 5′-flanking DNA, exons, and introns, whereas most of the 3′ CpG islands appeared to be associated with exons. CpG islands are generally found in the same position relative to the transcription unit of equivalent genes in different species, with some notable exceptions. CpG islands have been estimated to constitute I %-2% of the mammalian genome, and are found in the promoters of all housekeeping genes, as well as in a less conserved position in 40% of genes showing tissue-specific expression. The persistence of CpG dinucleotides in CpG islands is largely attributed to a general lack of methylation of CpG islands, regardless of expression status. The term “CpG site” refers to the CpG dinucleotide within the CpG islands. CpG islands are typically, but not always, between about 0.2 to about 1 kb in length.

As used herein, “detecting” refers to the identification of the presence or absence of a target nucleic acid molecule comprising the methylation marker in a urine sample. As used herein, “detecting” also refers to detecting the presence of the target nucleic acid molecule during a process wherein the signal generated by a directly or indirectly labeled probe nucleic acid molecule (capable of hybridizing to the target) is measured or observed. Thus, detection of the probe nucleic acid is directly indicative of the presence, and thus the detection, of a target nucleic acid, such as a sequence encoding a marker gene. For example, if the detectable label is a fluorescent label, the target nucleic acid is “detected” by observing or measuring the light emitted by the fluorescent label on the probe nucleic acid when it is excited by the appropriate wavelength, or if the detectable label is a fluorescence/quencher pair, the target nucleic acid is “detected” by observing or measuring the light emitted upon association or dissociation of the fluorescence/quencher pair present on the probe nucleic acid, wherein detection of the probe nucleic acid indicates detection of the target nucleic acid. If the detectable label is a radioactive label, the target nucleic acid, following hybridization with a radioactively labeled probe is “detected” by, for example, autoradiography. Methods and techniques for “detecting” fluorescent, radioactive, and other chemical labels maybe found in Ausubel et al. (1995, Short Protocols in Molecular Biology, 3r Ed. John Wiley and Sons, Inc.). Alternatively, a nucleic acid may be “indirectly detected” wherein a moiety is attached to a probe nucleic acid which will hybridize with the target, such as an enzyme activity, allowing detection in the presence of an appropriate substrate, or a specific antigen or other marker allowing detection by addition of an antibody or other specific indicator. Alternatively, a target nucleic acid molecule can be detected by amplifying a nucleic acid sample prepared from a patient clinical sample, using oligonucleotide primers which are specifically designed to hybridize with a portion of the target nucleic acid sequence. Quantitative amplification methods, such as, but not limited to TaqMan, may also be used to “detect” a target nucleic acid according to the invention. A nucleic acid molecule is “detected” as used herein where the level of nucleic acid measured (such as by quantitative PCR), or the level of detectable signal provided by the detectable label is at all above the background level.

As used herein, “detecting” further refers to detecting methylation state or status on a specific CpG site of a target nucleic acid molecule that is indicative of a pre-cancer or cancer condition of the individual and or the type of pre-cancer or cancer. The methylation state or status on a specific CpG site of a target nucleic acid molecule can provide useful information for diagnosis, disease monitoring, and therapeutic approaches. Various methods known in the art may be used for determining the methylation status of specific CpG dinucleotides. Such methods include but are not limited to methylated CpG island amplification, see Toyota et al., “Identification of differentially methylated sequences in colorectal cancer by methylated CpG island amplification,” Cancer Res., 59: 2307-2312 (1999), see also WO00/26401A1; differential methylation hybridization, see Huang et al., “Methylation profiling of CpG islands in human breast cancer cells,” Hum. Mol. Genet, 8: 459-470 (1999); methylation-specific PCR (MSP), see Herman et al, “Methylation-specific PCR: a novel PCR assay for methylation status of CpG islands,” PNAS USA 93: 9821-9826 (1992), see also U.S. Pat. No. 5,786,146; samethylation-sensitive single nucleotide primer extension (Ms-SnuPE), see U.S. Pat. No. 6,251,594; combined bisulfite restriction analysis (COBRA), see Xiong and Laird, “COBRA: a sensitive and quantitative DNA methylation assay,” Nucleic Acids Research, 25(12): 2532-2534 (1997); and methylation-specific primer extension (MSPE), etc.

A methylation marker is detected (present) when the marker CpG dinucleotide is methylated. A methylation marker is not detected (absent) when the marker CpG dinucleotide comprising the marker is not methylated.

A method of the invention comprises determining the presence or absence of one or more methylation marker of a methylation marker set in urine. A methylation marker is detected (present) when the marker CpG dinucleotide is methylated. A methylation marker is not detected (absent) when the marker CpG dinucleotide comprising the marker is not methylated. In a preferred embodiment the presence or absence of two or more methylation markers of a methylation marker set is determined. In a preferred embodiment the presence or absence of three or more, preferably four or more methylation markers of a methylation marker set are detected. The methylation marker set preferably comprises at least one methylation marker selected from the group consisting of the methylation markers identified in Table 9. The methylation marker set preferably comprises, and more preferably consists of the methylation marker set of Table 9. Determining the presence or absence of more than one methylation marker is helpful in case where the presence of one methylation marker is associated with more than one type of cancer or pre-cancer. In such cases more methylation markers are preferred. In such cases as many methylation markers as needed to discriminate between the cancers are preferably determined.

Detection of the presence of at least one methylation marker in a promoter region of at least one of the genes RASSF1A, ZNF154, TMEFF2, GDF15, CDKN2A, SHOX2, SOX17, GATA2, 3OST2, CYGB, FAM19A4, PHACTR3 and APC of Table 9 indicates that the individual has lung cancer, preferably non-small cell lung cancer. In a preferred embodiment at least one methylation marker in a promoter region of at least two of the genes RASSF1A, ZNF154, TMEFF2, CDKN2A, SHOX2, SOX17, GATA2, 3OST2, CYGB, FAM19A4, PHACTR3 and APC of Table 9 are detected to indicated that the individual has lung cancer, preferably non-small cell lung cancer. In a preferred embodiment methylation markers in a promoter region of at least three and preferably at least four of the genes RASSF1A, ZNF154, TMEFF2, CDKN2A, SHOX2, SOX17, GATA2, 3OST2. CYGB, FAM19A4, PHACTR3 and APC of Table 9 are detected to indicated that the individual has lung cancer, preferably non-small cell lung cancer. In case of determining whether an individual has lung cancer it is preferred that the presence or absence of a methylation marker in a promoter region of the gene RASSF1A is determined. In a preferred embodiment a methylation marker in a promoter region of the gene RASSF1A and at least one of the genes GATA2, 3OST2, GDF15, TMEFF2 is determined. In a preferred embodiment the presence or absence of a methylation marker in a promoter region of the gene RASSF1A and at least one, and preferably at least two, preferably at least three and more preferably four of the genes GATA2, 3OST2, GDF15, TMEFF2 is determined. In another embodiment of determining whether an individual has lung cancer it is preferred that the presence or absence of a methylation marker in a promoter region of the gene CYGB, FAM19A4 or PHACTR3 is determined. In another embodiment of determining whether an individual has lung cancer it is preferred that the presence or absence of a methylation marker in a promoter region of the gene CYGB is determined. In another embodiment of determining whether an individual has lung cancer it is preferred that the presence or absence of a methylation marker in a promoter region of the gene CYGB and at least one of FAM19A4 or PHACTR3 is determined. In a preferred embodiment a methylation marker in a promoter region of the gene CYGB and at least one of the genes GATA2, 3OST2, GDF15, TMEFF2 is determined. In a preferred embodiment the presence or absence of a methylation marker in a promoter region of the gene CYGB and at least one, and preferably at least two, preferably at least three and more preferably four of the genes GATA2, 3OST2, GDF15, TMEFF2, FAM19A4 or PHACTR3 is determined. Extending the panel of methylation markers increases the accuracy of the method.

Detection of the presence of at least one methylation marker in a promoter region of at least one of the genes GDF15, TMEFF2, VIM, TWIST1, NID2, ZNF154 and RASSF1A of Table 9 indicates that the individual has an Urothelial cell carcinoma such as bladder cancer. In a preferred embodiment at least one methylation marker in a promoter region of at least two of the genes GDF15, TMEFF2, VIM, TWIST1, NID2, ZNF154 and RASSF1A of Table 9 are detected to indicated that the individual has an Urothelial cell carcinoma such as bladder cancer. In a preferred embodiment methylation markers in a promoter region of at least three and preferably at least four of the genes GDF15; TMEFF2, VIM, TWIST1, NID2, ZNF154 and RASSF1A of Table 9 are detected to indicated that the individual has an Urothelial cell carcinoma such as bladder cancer. In case of determining whether an individual has an Urothelial cell carcinoma such as bladder cancer it is preferred that the presence or absence of a methylation marker in a promoter region of the gene GDF15 is determined. In a preferred embodiment a methylation marker in a promoter region of the gene GDF15 and at least one and preferably at least two, and more preferably three of the genes TMEFF2, VIM, and RASSF1A is determined.

Detection of the presence of at least one methylation marker in a promoter region of at least one of the genes CYGB, SFRP2A and MGMT of Table 9 indicates that the individual has an colon cancer. In a preferred embodiment at least one methylation marker in a promoter region of at least two of the genes CYGB, SFRP2A and MGMT of Table 9 are detected to indicate that the individual has colon cancer. In a preferred embodiment methylation markers in a promoter region of at least three of the genes CYGB, SFRP2A and MGMT of Table 9 are detected to indicated that the individual has colon cancer. In case of determining whether an individual has colon cancer it is preferred that the presence or absence of a methylation marker in a promoter region of the gene CYGB is determined. In a preferred embodiment methylation markers in a promoter region of at least two of the genes CYGB and MGMT of Table 9 are determined to indicated that the individual has colon cancer.

Extending the panel of methylation markers increases the accuracy of the method.

Detection of the presence of at least one methylation marker in a promoter region of at least one of the genes FAM19A4, PHACTR3, PRDM14, CAD M1, MAL and miR124-2 of Table 9 indicates that the individual has cervical cancer. In a preferred embodiment at least one methylation marker in a promoter region of at least two of the genes FAM19A4, PHACTR3, PRDM14, CAD M1, MAL and miR124-2 of Table 9 are detected to indicated that the individual has cervical cancer. In a preferred embodiment methylation markers in a promoter region of at least three and preferably at least four, preferably at least 5 of the genes, more preferably at least six of the genes FAM19A4, PHACTR3, PRDM14, CAD M1, MAL and miR124-2 of Table 9 are detected to indicated that the individual has cervical cancer. In case of determining whether an individual has cervical cancer it is preferred that the presence or absence of a methylation marker in a promoter region of the gene FAM19A4, the gene PRDM14 or preferably a combination thereof, is determined. It is preferred that the presence or absence of a methylation marker in a promoter region of the gene FAM19A4, the gene PRDM14 or preferably a combination thereof, is determined together with the presence or absence of a methylation marker in a promoter of one, two, three or preferably four of the genes MAL, miR124-2, PHACTR3 and CADM1. Extending the panel of methylation markers increases the accuracy of the method.

Detection of the presence of at least one methylation marker in a promoter region of the gene GSTP1 of Table 9 indicates that the individual has prostate cancer.

Some markers are detected in more than one tumor. In such cases the marker is particularly suited to indicate that an individual has cancer or pre-cancer. The type of (pre-) cancer can be determined by other diagnostic means and/or by including more methylation makers in the test.

A method of the invention may indicate that an individual has (pre-)cancer. In such a case the invention can often also indicate what the tissue of origin (the primary tumor site) of the cancer is. Indicating that an individual has a colon cancer does not mean that the cancer has to be located in the colon. The tumor can have metastasized and be present at a different location.

A method of the invention may indicate that an individual has cancer. More often though the method indicates that the tested individual has a significant chance of having cancer or pre-cancer. It is recommended that the individual has further tests to make the diagnosis cancer. In one aspect, a method of the invention is a selection method for individuals that have a higher than normal chance of having a pre-cancer or cancer.

Detection of the presence or absence of pre-cancer or cancer in an individual is preferably determined by determining the presence or absence of a methylation marker in a promoter region of a gene FAM19A4; RASSF1A or ZNF154 of table 9. In a preferred embodiment the detection of the presence or absence of pre-cancer or cancer in an individual is determined by determining the presence or absence of a methylation marker in a promoter region of a gene FAM19A4; RASSF1A of table 9 and a methylation marker in a promoter region of ZNF154 of table 9. and preferably the combination thereof. Determining that the methylations markers in a promoter region of a gene FAM19A4; or RASSF1A of table 9 and/or ZNF154 of table 9 are absent indicates that the individual from which the urine was collected, does not have, or does no longer have pre-cancer or cancer.

Detection of the presence or absence of methylation markers as disclosed in the present invention preferably comprises amplification of nucleic acid in the urine sample. Preferably detection of the presence of at least one methylation marker comprises amplification of nucleic acid of at least 15, preferably at least 30, preferably at least 50, preferably at least 100 and more preferably 150 consecutive nucleotides of a nucleic acid in the urine sample of the individual. Preferably at least one methylation marker is a CpG island in a promoter region of a gene identified in Table 9.

A methylation marker preferably comprises methylation of one or more CpG's in a CpG island in a promoter region of a gene listed in Table 9. A methylation marker of table 9 is thus methylation of one or more CpG's in a CpG island of a promoter region of a gene listed in table 9. One way of designing primers for methylation specific PCR (preferably quantitative PCR) to detect this methylation is discussed in Davidovic et al. and Snellenberg et al. [1, 2].

Determining a pre-cancer or cancer type preferably comprises detecting the presence of at least one methylation marker of Table 9 in the urine sample of the individual. The presence of a methylation marker of Table 9 indicates that the individual has cancer or pre-cancer. Subsequently the presence of a particular methylation marker typically includes certain pre-cancers or cancers in the typing. The absence of a certain other methylation marker can exclude a certain type from the list of types of pre-cancer or cancer. So both the presence and the absence can be indicative for certain types of pre-cancer or cancer. Determining that an individual does not have a cancer or a pre-cancer preferably comprises determining the absence of methylation markers in a promoter of two or more of the genes described in table 9. Preferably in a promoter of at least three, four, five, six, seven or more of the genes described in table 9.

The invention further provides a method for population screening of individuals for the occurrence of cancer or pre-cancer comprising collecting urine samples for said population of individuals and determining the presence or absence of one or more methylation markers of a methylation marker set said urine samples; and
determining whether there are individuals with cancer or pre-cancer based on the detection of the presence or absence of said one or more methylation markers in the urine samples, wherein the presence of said one or more methylation markers indicates that an individual of the population has pre-cancer or cancer and the absence of the methylation markers indicates that of the population does not have pre-cancer or cancer. The one or more methylation markers are preferably methylation markers of the invention, such as the methylation markers described in table 9.

Population screening, or testing a urine sample for determining whether an individual has cancer or pre-cancer is preferably performed on a urine sample or urine samples (in case of screening) of individual(s) of which it is not known what the cancer or pre-cancer status is.

The sample of the present invention is a urine sample. In a preferred embodiment the sample is a cell free urine sample. Cell free samples may be collected by filtration of urine or by centrifugation and collection of the supernatant. Other methods to essentially remove intact cells from a collected urine sample are known in the art and are also useful in the present invention. Typically, albeit not necessarily always, larger cell debris is also removed from the urine prior to performing a method of the invention. A cell as defined in the present invention is a cell that contains a nucleus. Erythrocytes can be present in urine and are preferably also removed. Cell free samples are suitably collected by harvesting supernatant after centrifugation at 3000×g.

The invention further provides a nucleic acid amplification device wherein preferably the methylation marker set comprises at least one means for detecting a methylation marker selected from the group consisting of the methylation markers identified in Table 9. In a preferred embodiment the device comprises nucleic acid amplification primers for amplifying nucleic acid comprising the methylation markers from the methylation marker set of Table 1.

### BRIEF DESCRIPTION OF THE DRAWINGS

FIG. 1: example of qPCR results of 2 methylation markers in urine of cancer patients (red) and urine of healthy controls (green). Boxplots are shown presenting the methylation level of respectively MGMT and FAM19A4 relative to the housekeeping gene B-actin on a logarithmic scale. MGMT is an example of a marker for which no discriminatory power between cancer patients and healthy controls was detected. FAM19A4 is an example of a marker with good discriminatory power.

FIG. 2: an example of a methylation specific qPCR results. Shown are both the amplification plots (top graph) of multiple genes and the product melting curves. Clearly seen is the different melting temperature for different amplicons (lower graph).

### EXAMPLES

### Materials & Methods

All urine samples were supplied by volunteers under full consent. Since the urine is a ‘waste product’ that is acquired non-invasively, no approval the medical and ethical overview board was required. Nevertheless, all subjects were asked permission for the use of their urine.

We collected urine of 20 patients with bladder cancer like symptoms, 20 patients with confirmed cervical cancer. In a first experiment we tested urine samples of 20 lung cancer patients and 10 healthy individuals. In a second experiment we tested 14 independent urine samples of lung cancer patients and 19 controls. We also tested urine samples of 11 patients with colon cancer and 22 control. For the cervical cancer patients routine methylation tests based on cervical swabs were compared to our results.

We investigate the promoter region methylation of the genes RASSF1A, VIM, GDF15 and TMEFF2 for bladder cancer, APC, GATA2, 3OST2, RASSF1A, GDF15, TMEFF2 VIM, GYGB, FAM19A4, and PHACTR3 for lung cancer, FAM19A4, PHACTR3, PRDM14, CAD M1, MAL and miR124-2 for cervical cancer, and CYGB, SFRP2A and MGMT for colon cancer.

TABLE 1
used primer sequences per gene in qPCR.

| Gene | Forward | Reverse |
|---|---|---|
| GDF15 | TCG GCG GTT ATT | CGT CGA AAA CAA |
| | TGT ATT TGC | CCG AAA CA |
| | (SEQ ID NO: 1) | (SEQ ID NO: 2) |
| TMEFF2 | GTT CGG GGT TAC | TTC GCC TCA CTC |
| | GCG C | TCC GCT |
| | (SEQ ID NO: 3) | (SEQ ID NO: 4) |
| VIM | TTC GGG AGT TAG | ACC GCC GAA CAT |
| | TTC GCG TT | CCT ACG A |
| | (SEQ ID NO: 5) | (SEQ ID NO: 6) |
| RASSF1A | GCG TTG AAG TCG | CCC GTA CTT CGC |
| | GGG TTC | TAA CTT TAA ACG |
| | (SEQ ID NO: 7) | (SEQ ID NO: 8) |
| GATA2 | GCG GTC GTT CGG | AAA CGA ACC GAA |
| | CGT GTC | CCG AAA ACG |
| | (SEQ ID NO: 9) | (SEQ ID NO: 10) |
| APC | GAA CCA AAA CGC | TTA TAT GTC GGT |
| | TCC CCA T | TAC GTG CGT TTA |
| | (SEQ ID NO: 11) | TAT |
| | | (SEQ ID NO: 12) |
| 3OST2 | TCG GCG TAC GTA | ATC TCC CGA TCC |
| | AGA GTT TGG | TAA ACG ATA AAA |
| | (SEQ ID NO: 13) | (SEQ ID NO: 14) |
| FAM19A4 | AGT CGG GCG GTT | CAA AAC GAC GCG |
| | CGG TT | CAA CT |
| | (SEQ ID NO: 15) | (SEQ ID NO: 16) |
| PHACTR3 | GGT TAT TTT GCG | CGA ATA CTC TAA |
| | AGC GGT TTC | TTC CAC GCG ACT |
| | (SEQ ID NO: 17) | (SEQ ID NO: 18) |
| PRDM14 | TTA CGT GTT ATT | ATC TAT TCC TAA |
| | GTC GGG GAT T | TAC CTA AAA ACG |
| | (SEQ ID NO: 19) | AAA CG |
| | | (SEQ ID NO: 20) |
| CADM1 | CGT ATG TTA TTA | CGC TCG ACA ACA |
| | GTA TTT TAT TAG | CTA CAC TCG |
| | TTG TTC GTT C | (SEQ ID NO: 22) |
| | (SEQ ID NO: 21) | |
| MAL | GGT TAT TGG GTT | GTA CTA ACG TCG |
| | TCG CG | ACC TTA AAA CGA |
| | (SEQ ID NO: 23) | (SEQ ID NO: 24) |
| mir124-2 | GGT AAT TAA TTT | CGT AAA AAT ATA |
| | GGA TTT ACG TCG | AAC GAT ACG TAT |
| | TTA T | ACC TAC GT |
| | (SEQ ID NO: 25) | (SEQ ID NO: 26) |
| β-actin | TGG TGA TGG AGG | AAC CAA TAA AAC |
| | AGG TTT AGT AAG | CTA CTC CTC CCT |
| | T | TAA |
| | (SEQ ID NO: 27) | (SEQ ID NO: 28) |
| CYGB | CGA GGT CGA TCG | CCA ACG ACT AAC |
| | TTA GTT CGT TC | TCG AAA ACG CG |
| | (SEQ ID NO: 29) | (SEQ ID NO: 30) |
| SFRP2 | CGT TTT AGT CGT | TCC CGA ACC CGC |
| | CGG TTG TTA GTT | TCT CTT |
| | T | (SEQ ID NO: 32) |
| | (SEQ ID NO: 31) | |
| MGMT | GAT TTT TAT TAA | CTT TTC CTA TCA |
| | GCG GGC GTC | CAA AAA TAA TCC |
| | (SEQ ID NO: 33) | G |
| | | (SEQ ID NO: 34) |

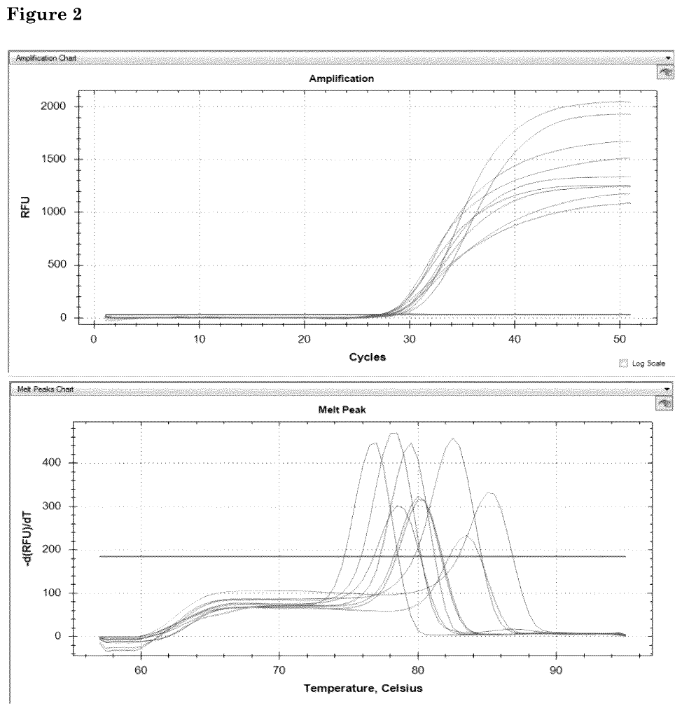

DNA was extracted from the urine samples (5-40 mL urine per extraction, dependent on availability) with the Zymo research ‘Quick-DNA™ Urine’ kit according to protocol. The DNA was subsequently treated with bisulfite (Zymo research ‘EZ DNA Methylation™’ Kit according to protocol). The resulting bisulfite treated DNA was used for methylation specific qPCR (MSP) in a BIORAD real time CFX 384, using iQ SYBR Green supermix or on an ABI-7500 real-time PCR-system (Applied Biosystems). The results were analyzed using machine specific software (see FIG. 2 for a data example).

### Results

With respect to blood, urine already contains a concentrate of the ‘waste products’ of the body, due to initial filtration and consecutive active resorption of useful compounds. Furthermore, urine is available in greater quantities than blood, allowing us to start with up to 40 mL urine. By eluting the DNA from that urine in 10 μL H₂O a concentration step of 4000× is achieved on top of the concentration done by the kidneys prior to quantitative PCR (qPCR).

All qPCR samples were simultaneously tested for the presence of the gene β-actin, which is used here as a methylation independent marker. This was used to assess whether enough DNA for qPCR was present in the urine sample. Some samples do not contain (sufficient) DNA.

The qPCR results of the bladder cancer samples showed 12 out of 20 with enough DNA in the sample and out of these 12, 11 were positive for one or more of our methylation markers.

TABLE 2
genes detected in the urine of bladder cancer patients with sufficient DNA
concentrations for MSP analysis. These urine samples were tested for
GDF15, TMEFF2, VIM and RASSF1A.

| | |
|---|---|
| Sample # | Detected gene methylation |

| | | | |
|---|---|---|---|
| 2 | GDF15 | TMEFF2 | RASSF1A |
| 3 | GDF15 | TMEFF2 | VIM |
| 7 | GDF15 | TMEFF2 | RASSF1A |
| 8 | GDF15 | TMEFF2 | |
| 11 | GDF15 | TMEFF2 | |
| 12 | — | | |
| 14 | GDF15 | TMEFF2 | |
| 15 | GDF15 | TMEFF2 | |
| 17 | GDF15 | TMEFF2 | VIM |
| 18 | GDF15 | TMEFF2 | VIM |
| 19 | GDF15 | TMEFF2 | VIM |
| 20 | GDF15 | TMEFF2 | |

The urines of cervical cancer patients were used to compare the methylation patterns of swabs with spontaneous urines and catheter urines. Similar methylation patterns were be seen in the urine and swab samples. The catheter urine (which has no contact with tumor cells or tumor cell debris) showed clear methylation of our marker genes as seen in table 3 below. The 5 healthy female control subjects were all negative for all 6 markers in urine.

TABLE 3
genes detected in the urine of cervical cancer patients compared to cervical swabs. Not all samples
were available for each patient. The reference to FAM is a reference to the gene FAM19A4.

| Patient | sample type | DNA Control | FAM | PHACTR3 | PRDM14 | CADM1 | MAL | miR124-2 |
|---|---|---|---|---|---|---|---|---|
| #1 | Swab | ok | FAM | PHACTR3 | PRDM14 | n.a. | n.a. | n.a. |
| | urine (catheter) | ok | — | — | — | — | — | — |
| #2 | Swab | not sufficient | — | — | — | n.a. | n.a. | n.a. |
| | urine (catheter) | ok | FAM | — | PRDM14 | — | — | — |
| #3 | Swab | not sufficient | — | — | — | n.a. | n.a. | n.a. |
| | urine (catheter) | ok | — | — | PRDM14 | — | MAL | — |
| #4 | Swab | ok | FAM | PHACTR3 | PRDM14 | n.a. | n.a. | n.a. |
| | urine (catheter) | ok | FAM | PHACTR3 | PRDM14 | CADM1 | MAL | miR124-2 |
| #5 | Swab | ok | — | — | — | n.a. | n.a. | n.a. |
| | urine (catheter) | ok | — | — | PRDM14 | — | — | — |
| #6 | Swab | not sufficient | — | — | — | n.a. | n.a. | n.a. |
| | urine (catheter) | not sufficient | — | — | — | n.a. | n.a. | n.a. |
| #7 | Swab | not sufficient | — | — | — | n.a. | n.a. | n.a. |
| | urine (catheter) | ok | FAM | — | PRDM14 | CADM1 | MAL | miR124-2 |
| #8 | Swab | ok | FAM | — | — | n.a. | n.a. | n.a. |
| | urine (catheter) | ok | FAM | — | PRDM14 | — | MAL | — |
| #9 | Swab | not sufficient | — | — | — | n.a. | n.a. | n.a. |
| | urine (catheter) | ok | — | — | — | — | MAL | miR124-2 |
| #10 | Swab | not sufficient | — | — | — | n.a. | n.a. | n.a. |
| | urine (catheter) | ok | FAM | — | PRDM14 | — | — | — |
| | Urine (spontaneous) | ok | FAM | — | PRDM14 | — | — | — |
| #11 | Swab | not sufficient | — | — | — | n.a. | n.a. | n.a. |
| | Urine (spontaneous) | not sufficient | — | — | — | n.a. | n.a. | n.a. |
| #12 | Swab | not sufficient | — | — | — | n.a. | n.a. | n.a. |
| | urine (catheter) | ok | FAM | PHACTR3 | PRDM14 | — | MAL | miR124-2 |
| | Urine (spontaneous) | ok | FAM | PHACTR3 | PRDM14 | — | MAL | miR124-2 |
| #13 | Swab | not sufficient | — | — | — | n.a. | n.a. | n.a. |
| | urine (catheter) | ok | FAM | — | PRDM14 | — | MAL | miR124-2 |
| | Urine (spontaneous) | ok | FAM | — | PRDM14 | — | MAL | miR124-2 |
| #14 | Swab | not sufficient | — | — | — | n.a. | n.a. | n.a. |
| | urine (catheter) | ok | FAM | PHACTR3 | PRDM14 | CADM1 | MAL | miR124-2 |
| #15 | Swab | not sufficient | — | — | — | n.a. | n.a. | n.a. |
| | urine (catheter) | ok | FAM | PHACTR3 | PRDM14 | — | — | — |
| | Urine (spontaneous) | ok | FAM | PHACTR3 | PRDM14 | — | MAL | miR124-2 |
| #16 | Swab | not sufficient | — | — | — | n.a. | n.a. | n.a. |
| | Urine (spontaneous) | ok | — | PHACTR3 | — | — | — | — |
| #17 | Swab | not sufficient | — | — | — | n.a. | n.a. | n.a. |
| | urine (catheter) | ok | — | — | PRDM14 | — | — | — |
| | Urine (spontaneous) | ok | FAM | PHACTR3 | PRDM14 | CADM1 | MAL | miR124-2 |
| #18 | Swab | not sufficient | — | — | — | n.a. | n.a. | n.a. |
| | urine (catheter) | ok | FAM | — | PRDM14 | — | MAL | — |
| | Urine (spontaneous) | ok | FAM | PHACTR3 | — | CADM1 | MAL | miR124-2 |
| #19 | Swab | not sufficient | — | — | — | n.a. | n.a. | n.a. |
| | urine (catheter) | ok | FAM | — | PRDM14 | — | — | — |
| #20 | Swab | not sufficient | — | — | — | n.a. | n.a. | n.a. |
| | urine (catheter) | ok | — | PHACTR3 | — | — | — | miR124-2 |
| | Urine (spontaneous) | ok | FAM | PHACTR3 | PRDM14 | — | — | — |

The presence of methylated DNA sequences in the catheter urine of the cervical cancer patients indicates the ability of the methylation markers to reach the urine without direct contact of the urine with the tumor, because catheter urine does not pass the vulva which excludes cervical, vaginal and vulvar contamination. To confirm that tumor-derived methylated DNA sequences reach the urine without direct contact, a non-genitourinary cancer was investigated excluding the possibility of ex vivo contamination: lung cancer. 2 sets of patient samples were analyzed with 2 different methylation marker panels.

Only patients with a positive signal on the □-actin control gene are listed in table 4. In the first sample set, we found enough DNA (□-actin methylation independent control) in 19 out of 27 samples. Samples were tested for RASSF1A, GATA2, 3OST2, APC, GDF15, TMEFF2, and VIM. Of these 19 samples 11 showed hypermethylation of the promoter regions of the genes named below.

TABLE 4
methylated markers found in urine from lung cancer patients.

| patient | positive genes | | | | |
|---|---|---|---|---|---|
| 1 | — | | | | |
| 2 | RASSF1A | APC | GATA2 | 3OST2 | TMEFF2 |
| 3 | RASSF1A | GATA2 | GDF15 | | |
| 4 | RASSF1A | | | | |
| 5 | RASSF1A | | | | |
| 6 | RASSF1A | TMEFF2 | | | |
| 10 | — | | | | |
| 11 | — | | | | |
| 12 | GDF15 | | | | |
| 14 | RASSF1A | | | | |
| 15 | — | | | | |
| 16 | — | | | | |
| 18 | RASSF1A | | | | |
| 20 | RASSF1A | | | | |
| 21 | 3OST2 | | | | |
| 22 | — | | | | |
| 23 | — | | | | |
| 24 | 3OST2 | VIM | GDF15 | | |
| 26 | — | | | | |

Out of the 10 healthy controls in this sample set, 8 had a positive □-actin signal. Of these 8 samples 2 showed methylation positivity for one or two markers (table 5), indicating the necessity of cut-off values for this test.

TABLE 5
control samples tested in combination with the lung cancer samples.

| Control | | |
|---|---|---|
| 2 | — | |
| 3 | — | |
| 4 | — | |
| 5 | — | |
| 6 | RASSF1A | GDF15 |
| 7 | — | |
| 9 | RASSF1A | |
| 10 | — | |

In the second sample set, 14 patient samples had a positive b-actin signal. Of these 14 samples, 11 showed hypermethylation of the promoter regions of 3 genes named CYGB, FAM19A4 and PHACTR3 above a certain threshold value (table 6). In zero out of 19 healthy control samples with a positive b-actin signal, these 3 markers were detected above the same threshold. A fourth methylation marker investigated: PRDM14 was identified in 12 of the 14 patient samples, but was also identified in 8 out of the 19 control samples (Table 7). This indicates that not all hypermethylated DNA sequences previously identified in tumors have a strong diagnostic power in urine samples.

TABLE 6
methylated markers found in urine from lung cancer patients.

| | |
|---|---|
| patient | postive genes |

| | | | | |
|---|---|---|---|---|
| 1 | | FAM19A4 | | PRDM14 |
| 3 | | FAM19A4 | PHACTR3 | PRDM14 |
| 4 | — | | | PRDM14 |
| 5 | CYGB | FAM19A4 | | PRDM14 |
| 6 | CYGB | FAM19A4 | | PRDM14 |
| 8 | — | | | |
| 9 | | FAM19A4 | | |
| 10 | | | PHACTR3 | PRDM14 |
| 11 | — | | | PRDM14 |
| 13 | CYGB | | | PRDM14 |
| 15 | | | PHACTR3 | PRDM14 |
| 16 | CYGB | | | PRDM14 |
| 17 | | FAM19A4 | | PRDM14 |
| 18 | | FAM19A4 | | PRDM14 |

TABLE 7
methylated markers found in urine from healthy controls.

| control | | |
|---|---|---|
| 1 | — | PRDM14 |
| 2 | — | PRDM14 |
| 3 | — | |
| 4 | — | |
| 5 | — | |
| 6 | — | PRDM14 |
| 8 | — | PRDM14 |
| 9 | — | PRDM14 |
| 10 | — | |
| 11 | — | |
| 12 | | PRDM14 |
| 13 | — | |
| 14 | — | |
| 16 | — | |
| 17 | — | |
| 19 | — | |
| 20 | — | |
| 21 | — | PRDM14 |
| 22 | — | PRDM14 |

To further establish the conclusion that at least some hypermethylated DNA sequences from tumors that are not in contact with urine do reach the urine and can be detected in urine samples of patients, we tested another set of urine samples from patients with a non-genitourinary cancer: colon cancer.

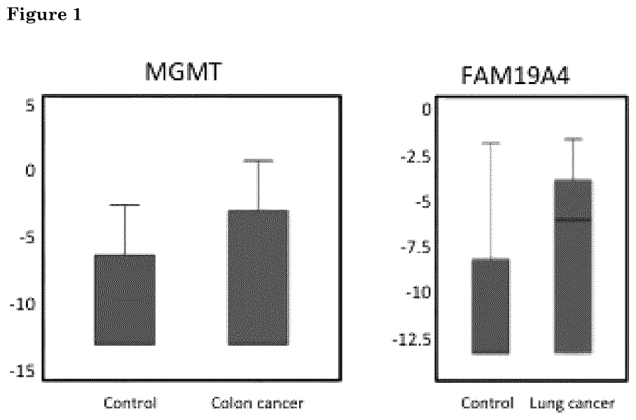

11 urine samples had a positive b-actin signal. These samples were tested for 3 methylation markers: CYGB, SFRP2 and MGMT. All of these markers were detectable in the urine of cancer patients. Of these 3 markers only CYGB was more often detected in the urine of patients compared to healthy controls (see table 7). The absence of discriminatory power for MGMT is shown in FIG. 1. This again underscores that not all methylated DNA sequences identified in tumors can be used as cancer-selective markers in urine samples.

TABLE 8
methylated markers found in urine from colon cancer patients and healthy
controls.

| patient | postive genes | | control | | |
|---|---|---|---|---|---|
| 1 | | SFRP2 | 1 | | SFRP2 |
| 2 | CYGB | SFRP2 | 2 | | SFRP2 |
| 5 | | | 3 | | SFRP2 |
| 6 | CYGB | | 4 | | SFRP2 |
| 7 | | | 5 | | SFRP2 |
| 8 | | | 6 | | SFRP2 |
| 9 | | SFRP2 | 8 | | SFRP2 |
| 11 | | SFRP2 | 9 | | SFRP2 |
| 13 | | SFRP2 | 10 | — | |
| 17 | | | 11 | — | |
| 20 | CYGB | | 12 | | SFRP2 |
| | | | 13 | — | |
| | | | 14 | | SFRP2 |
| | | | 16 | — | |
| | | | 17 | — | |
| | | | 19 | — | |
| | | | 20 | | SFRP2 |
| | | | 21 | | SFRP2 |
| | | | 22 | | SFRP2 |

FIG. 1 shows an example of qPCR results of 2 methylation markers in urine of cancer patients (red) and urine of healthy controls (green). Boxplots are shown presenting the methylation level of respectively MGMT and FAM19A4 relative to the housekeeping gene B-actin on a logarithmic scale. MGMT is an example of a marker for which no discriminatory power between cancer patients and healthy controls was detected. FAM19A4 is an example of a marker with good discriminatory power.

### CONCLUSION

The results show that urine contains sufficient tumor DNA that retains sufficient of its methylation status for detection, even if the urine has no direct contact with the tumor(cells). Furthermore the detected methylated DNA fragments can be linked to the gene specific sequences that are indicative for tumor detection and prediction. Therefore we conclude that the detection of cell free methylated DNA fragments in urine is a viable method for cancer detection and typing.

TABLE 9
Methylation markers of the present invention are present in the CpG
islands of the promoter region(s) of the genes indicated in this table. Primers for
methylation specific PCR (MSP) can be designed according to guidelines in
Davidovic et al and Snellenberg et al [1, 2].

| Gene name | Gene ID # | Location | gene description |
|---|---|---|---|
| APC | 324 | Chromosome 5, NC_000005.10 | adenomatous |
| | | (112707505 . . . 112846239) | polyposis coli |
| GATA2 | 2624 | Chromosome 3, NC_000003.12 | GATA binding protein 2 |
| | | (128479422 . . . 128493187, complement) | |
| 3OST2 | 9956 | Chromosome 16, NC_000016.10 | heparan sulfate- |
| | | (22814203 . . . 22916338) | glucosamine 3- |
| | | | sulfotransferase 2 |
| RASSF1A | 11186 | Chromosome 3, NC_000003.12 | Ras association |
| | | (50329786 . . . 50340936, complement) | domain family |
| | | | member 1 |
| GDF15 | 9518 | Chromosome 19, NC_000019.10 | growth differentiation |
| | | (18386158 . . . 18389176) | factor 15 |
| TMEFF2 | 23671 | Chromosome 2, NC_000002.12 | transmembrane |
| | | (191949046 . . . 192194940, complement) | protein with EGF like |
| | | | and two follistatin |
| | | | like domains 2 |
| VIM | 7431 | Chromosome 10, NC_000010.11 | vimentin |
| | | (17227935 . . . 17237593) | |
| GSTP1 | 2950 | Chromosome 11, NC_000011.10 | glutathione S- |
| | | (67583595 . . . 67586653) | transferase pi 1 |
| FAM19A4 | 151647 | Chromosome 3, NC_000003.12 | family with sequence |
| | | (68731764 . . . 68932610, complement) | similarity 19 |
| | | | (chemokine (C-C |
| | | | motif)-like), member |
| | | | A4 |
| PHACTR3 | 116154 | Chromosome 20, NC_000020.11 | phosphatase and |
| | | (59577509 . . . 59847711) | actin regulator 3 |
| PRDM14 | 63978 | Chromosome 8, NC_000008.11 | PR domain 14 |
| | | (70051126 . . . 70071327, complement) | |
| CADM1 | 23705 | Chromosome 11 NC_000011.10 | cell adhesion |
| | | (115173625 . . . 115504523) | molecule 1 |
| MAL | 4118 | Chromosome 2, NC_000002.12 | mal T-cell |
| | | (95025655 . . . 95053992) | differentiation protein |
| MIR124-2 | 406908 | Chromosome 8, NC_000008.11 | microRNA 124-2 |
| | | (64379149 . . . 64379257) | |
| SHOX2 | 6474 | Chromosome 3, NC_000003.12 | short stature |
| | | (158096011 . . . 158106202, complement) | homeobox 2 |
| CDKN2A | 1029 | Chromosome 9, NC_000009.12 | cyclin-dependent |
| | | (21967752 . . . 21995043, complement) | kinase inhibitor 2A |
| SOX17 | 64321 | Chromosome 8, NC_000008.11 | SRY-box 17 |
| | | (54457935 . . . 54460896) | |
| TWIST1 | 7291 | Chromosome 7, NC_000007.14 | twist family bHLH |
| | | (19113047 . . . 19117672, complement) | transcription factor 1 |
| NID2 | 22795 | Chromosome 14, NC_000014.9 | nidogen 2 |
| | | (52004802 . . . 52069231, complement) | |
| ZNF154 | 7710 | Chromosome 19, NC_000019.10 | zinc finger protein |
| | | (57696275 . . . 57709211, complement) | 154 |
| CYGB | 114757 | Chromosome 17, NC_000017.11 | cytoglobin |
| | | (76527348 . . . 76557692, complement) | |
| SFRP2 | 6423 | Chromosome 4, NC_000004.12 | secreted frizzled |
| | | (153780590 . . . 153789076, complement) | related protein 2 |
| MGMT | 4255 | Chromosome 10, NC_000010.11 | O-6-methylguanine- |
| | | (129467184 . . . 129770983) | DNA |
| | | | methyltransferase |

### CITED ART

1. Snellenberg, S., et al., Development of a multiplex methylation-specific PCR as candidate triage test for women with an HPV-positive cervical scrape. BMC Cancer, 2012. 12: p. 551.
2. Davidovic, R. S., et al., Methylation-specific PCR: four steps in primer design. Central European Journal of Biology, 2014. 9(12): p. 1127-1139.

1. An in vitro method of determining whether an individual has a pre-cancer or cancer comprising
determining the presence or absence of one or more methylation markers of a methylation marker set in a urine sample of said individual; and
determining whether the individual has pre-cancer or cancer based on the detection of the presence or absence of said one or more methylation markers in the urine sample, wherein the presence of said one or more methylation markers indicates that the individual has pre-cancer or cancer and the absence of the methylation markers indicates that the individual does not have pre-cancer or cancer.

2. An in vitro method of determining a type of pre-cancer or cancer in an individual that has cancer comprising
determining the presence or absence of one or more methylation markers of a methylation marker set in a urine sample of said individual; and
determining pre-cancer or cancer type(s) based on the detection of the presence or absence of said one or more methylation markers in the urine sample, wherein the presence or absence of said one or more methylation markers is indicative for one or more types of pre-cancer or cancer of said individual.

3. The method of claim 1 or claim 2, the methylation marker set comprises at least one methylation marker selected from the group consisting of the methylation markers identified in Table 9.

4. The method of any one of claims 1-3, wherein
detection of the presence of at least one of the methylation markers in a promoter region of RASSF1A, ZNF154, TMEFF2, GDF15, CDKN2A, SHOX2, SOX17, GATA2, 3OST2; GYGB; FAM19A4; PHACTR3 and APC of Table 9 indicates that the individual has lung cancer, preferably non-small cell lung cancer;
detection of the presence of at least one of the methylation markers in a promoter region of at least one of the genes GDF15; TMEFF2, VIM, TWIST1, NID2, ZNF154 and RASSF1A of Table 9 indicates that the individual has a Urothelial cell carcinoma such as bladder cancer;
detection of the presence of at least one of the methylation markers GSTP1 of Table 9 indicates that the individual has prostate cancer;
detection of the presence of at least one methylation marker in a promoter region of at least one of the genes FAM19A4, PHACTR3, PRDM14, CADM1, MAL and miR124-2 of Table 9 indicates that the individual has cervical cancer; and
detection of the presence of at least one methylation marker in a promoter region of at least one of the genes CYGB, SFRP2A and MGMT of Table 9 indicates that the individual has colon cancer.

5. The method of claim 1-4, wherein detection of the presence or absence of methylation markers comprises amplification of nucleic acid in the urine sample.

6. The method of claim 1-5, wherein at least one methylation marker is a CpG island in a promoter region of a gene identified in Table 9.

7. The method of claims 1-6, wherein the pre-cancer is selected from the group consisting of actinic keratosis; Barrett's esophagus; atrophic gastritis; dyskeratosis congenital; sideropenic dysphagia; lichen planus; oral submucous fibrosis; solar elastosis; cervical dysplasia; leukoplakia; polyposis coli and erythroplakia and/or the cancer is selected from the group consisting of lung cancer, preferably non-small cell lung cancer, cervical cancer, prostate cancer, ovarian carcinoma, breast cancer, including ductal carcinoma in situ, head and neck cancer, Urothelial cell carcinoma such as bladder cancer, Hematological malignancies such as but not limited to myeloma, leukemia, lymphoma and myelodysplastic syndrome, a cancer of the gastrointestinal tract (GI tract) and accessory organs of digestion, including but not limited to the esophagus, stomach, biliary system, pancreas, small intestine, large intestine, gall bladder cancer, liver cancer, anal cancer and rectum.

8. The method of claim 1-7, wherein determining a pre-cancer or cancer type comprises detecting the absence of at least one methylation marker of Table 9 in the urine sample of the individual.

9. The method of claim 1-8, wherein the urine sample is a cell free urine sample.

10. A kit comprising means for the detection of one or more methylation markers in a methylation marker set wherein the set comprises at least one methylation marker selected from the group consisting of the methylation markers identified in Table 9 and wherein the set further comprises an nucleic acid extraction buffer for extraction of nucleic acid from urine.

11. A nucleic acid amplification device comprising at least two nucleic acid amplification primers for amplifying nucleic acid of one or more methylation markers from a methylation marker set.

12. A nucleic acid amplification device according to claim 11, wherein the methylation marker set comprises at least one methylation marker selected from the group consisting of the methylation markers identified in Table 9.

13. A nucleic acid amplification device according to claim 11 or 12, comprising nucleic acid amplification primers for amplifying nucleic acid comprising the methylation markers from the methylation marker set of Table 9.

14. Use of kit according to claim 10 or a device according to any one of claims 11-13, for determining the type of pre-cancer or cancer of an individual with cancer from a cell free urine sample of the individual.

15. Use of kit according to claim 10 or a device according to any one of claims 11-13, for detecting a pre-cancer or cancer in an individual from a cell free urine sample of the individual.
